(19) United States
(12) Patent Application Publication
(10) Pub. No.: US 2017/0296393 A1
(43) Pub. Date: Oct. 19, 2017

(54) SKIN ADHESIVE MEMBER

(71) Applicant: 3M INNOVATIVE PROPERTIES COMPANY, SAINT PAUL, MN (US)

(72) Inventors: JOICHI TAGAMI, OGAKI-SHI, GIFU PREFECTURE (JP); NOBORU KODA, SETAGAYA-KU, TOKYO (JP)

(73) Assignee: 3M INNOVATIVE PROPERTIES COMPANY, SAINT PAUL, MN (US)

(21) Appl. No.: 15/516,741

(22) PCT Filed: Oct. 5, 2015
(86) PCT No.: PCT/US2015/053913

§ 371 Date: Apr. 4, 2017

(30) Foreign Application Priority Data
Oct. 10, 2014 (JP) 2014-209159

(51) Int. Cl. A61F 13/02; A61F 13/14; A61F 5/02

(52) U.S. Cl. CPC A61F 13/02; A61F 13/148; A61F 13/14; A61F 5/028

(57) ABSTRACT

A skin adhesive member that helps a user maintain an appropriate posture while supporting muscles on the abdominal side. The skin adhesive member extends in a length direction and is adhered from an abdominal side to a back side across a side abdomen of the user, the skin adhesive member having a foundation portion, a first extension portion extending in the length direction from one end of the foundation portion and adhered to the back side, a second extension portion extending from another end of the foundation portion and adhered to the abdominal side, wherein the first extension portion includes at least a pair of belt portions.

### TECHNICAL FIELD

The present invention relates to a skin adhesive member.

### BACKGROUND

Conventionally, a skin adhesive member is known for supporting a body by adhering to the body of a user (for example, refer to Registration of Design Publication No. 1364663). The skin adhesive member disclosed in Registration of Design Publication No. 1364663 supports by being adhered to the torso of the user (here, chest/lower back portion).

### SUMMARY

It is common among manual laborers to injure their lower back while working, and there are also many cases of injuring other portions of the body as a result of taking an unreasonable posture to avoid injuring the lower back when lifting a heavy object. To avoid this obstacle, it is important to not curve the upper body and lift without bending the lower back while keeping the upper body straight.

Therefore, a skin adhesive member is sought that helps the user easily maintain an appropriate posture while supporting muscles on the abdominal side.

The skin adhesive member according to one aspect of the present invention is a skin adhesive member extending in a length direction, adhered from an abdominal side to a back side across a side abdomen, being provided with a foundation portion having one end portion and an other end portion in the length direction, a first extension portion formed extending in the length direction from the one end portion of the foundation portion, and adhered to the back side, a second extension portion formed extending in the length direction from the other end portion of the foundation portion, and adhered to the abdominal side, wherein the first extension portion includes at least a pair of belt portions provided aligned in a width direction perpendicular to the length direction.

According to this skin adhesive member, because the first extension portion is adhered on the back side, the muscles of the back side can be supported. Furthermore, because the second extension portion is adhered on the abdomen side, the muscles of the abdominal side can be supported. Here, because the first extension portion includes at least one pair of belt portions, when this pair of belt portions is extended in the length direction, adhering to the back side so that the separation distance gets larger toward the tip end portion side in the width direction allows a stress to be generated so that the separation distance in the width direction (standing direction) gets closer. By this, the contraction of erector muscles is supported on the back side, and it can be easier for the user to stand erect. By the above, it is easy for the user to maintain an appropriate posture while supporting muscles on the abdominal side.

The skin adhesive member according to another aspect of the present invention extending in a length direction, adhered from an abdominal side to a back side across a side abdomen, being provided with a foundation portion having one end portion and an other end portion in the length direction, a first extension portion formed extending in the length direction from the one end portion of the foundation portion, and adhered to the back side, a second extension portion formed extending in the length direction from the other end portion of the foundation portion, and adhered to the abdominal side, wherein the first extension portion has a prescribed length in the length direction and includes a pair of edge portions opposing each other in a width direction perpendicular to the length direction, and the pair of edge portions have separation distances in the width direction that are larger towards the tip end portion side of the first extension portion.

In the skin adhesive member according to another aspect, the first extension portion may include a coupling portion that couples a pair of edge portions on a tip end portion.

In the skin adhesive member according to another aspect, a through-hole may be formed on a region surrounded by the one end portion of the foundation portion, the pair of edge portions, and the coupling portion on the first extension portion.

In the skin adhesive member according to another aspect, an adhesive material may be spread on the entirety of a region surrounded by the one end portion of the foundation portion, the pair of edge portions, and the coupling portion on the first extension portion.

The skin adhesive member according to another aspect, being further provided with a peeling member that protects an adhesive face of the skin adhesive member, wherein the peeling member includes a separation portion formed on the border region of the foundation portion and the first extension portion, and the separation portion may have a portion on the external side in the width direction positioned more to the foundation portion side than the portion on the internal side in the width direction.

In the skin adhesive member according to another aspect, the elasticity in the length direction of the first extension portion may be higher than the elasticity in the width direction.

### BRIEF DESCRIPTION OF THE DRAWINGS

FIG. 1 is a front view of the skin adhesive member of embodiment 1.

FIG. 2 is a back view of the skin adhesive member illustrated in FIG. 1.

FIG. 3 is a diagram illustrating an application example of the skin adhesive member illustrated in FIG. 1.

FIG. 4 is a diagram illustrating an application example of the skin adhesive member illustrated in FIG. 1.

FIG. 5 is a diagram illustrating an application example of the skin adhesive member illustrated in FIG. 1.

FIG. 6 is a front view of the skin adhesive member of embodiment 2.

FIG. 7 is a back view of the skin adhesive member illustrated in FIG. 6.

FIG. 8 is a front view of the skin adhesive member of embodiment 3.

FIG. 9 is a back view of the skin adhesive member illustrated in FIG. 8.

FIG. 10 is a front view of the skin adhesive member of embodiment 4.

FIG. 11 is a back view of the skin adhesive member illustrated in FIG. 10.

### DETAILED DESCRIPTION

Skin adhesive members of the present invention can make it easy for the user to maintain an approprtate posture while supporting muscles on the abdominal side. Embodiments of the present invention will be described in detail with reference to the accompanying drawings. Note that the same or similar components in the description of the drawings are given the same numerals, and overlapping descriptions are omitted.

### Embodiment 1

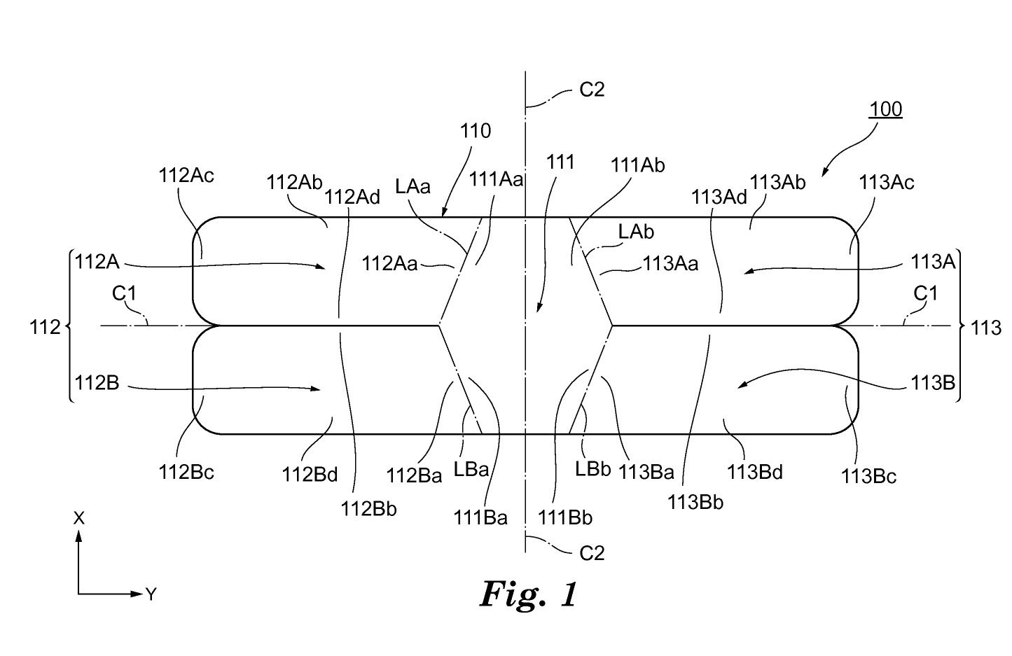

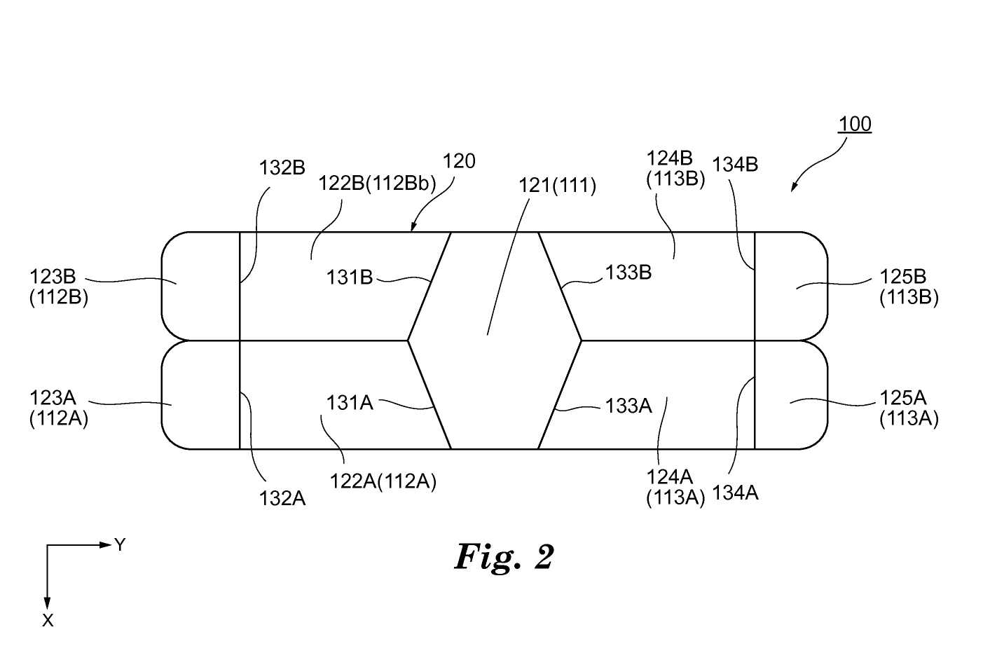

First, the configuration of a skin adhesive member 100 according to embodiment 1 will be described with reference to FIG. 1 and FIG. 2. FIG. 1 is a front view of the skin adhesive member 100 viewed from the opposite side of an adhesive face (described later), and FIG. 2 is a back view of the skin adhesive member 100 viewed from the adhesive face. FIG. 1 and FIG. 2 illustrate the skin adhesive member 100 prior to adhering to the body of the user. The skin adhesive member 100 is a tape adhered to support human muscles. The skin adhesive member 100 is made up of a double structure by a tape body 110 and a peeling sheet 120.

The tape body 110 is formed by applying an adhesive to one face (adhesive face) of a woven fabric. For example, the tape body 110 can be created by applying an acrylic, silicone, or rubber adhesive on one face of a woven fabric created by a mixture of cotton and urethane. Naturally, the woven fabric, resin and adhesive that configure the tape body 110 are not limited to these, and any material may be used for each.

The peeling sheet 120 is a sheet-shaped peeling member adhered on the adhesive face to protect the adhesive face of the tape body 110. A silicon process may be performed on the peeling sheet 120 to make it easier to remove the peeling sheet from the adhesive face.

Because the tape body 110 and the peeling sheet 120 are the same shape entirely, the shape of the tape body 110 will be described below.

As illustrated in FIG. 1, the tape body 110 is long along one direction, this direction is referred to as the length direction Y along the Y-axis in the present specification, and a direction perpendicular to this length direction is referred to as the width direction X along the X-axis. Furthermore, there are times when the right side of the paper surface in FIG. 1 is referred to as the “Y-axis positive direction”, the left side of the paper surface is referred to as the “Y-axis negative direction”, the upper side of the paper surface is referred to as the “X-axis positive direction”, and the lower side of the paper surface is referred to as the “X-axis negative direction”. The dimensions of the entirety of the tape body 110 may be arbitrarily determined based on the physique of the user and the like, and may have, for example, a length of 230 mm and a width of 75 mm. However, the dimensions are not limited to these, and may be, for example, a length of 150 to 400 mm, and a width of 30 to 150 mm.

The tape body 110 has a substantially rectangular shape for the entirety, and is provided with three portions of a foundation portion 111, a first extension portion 112, and a second extension portion 113 along the length direction Y. In FIG. 1, borders LAa, and LBa between the foundation portion 111 and the first extension portion 112 are illustrated with dashed lines and borders LAb, and LBb between the foundation portion 111 and the second extension portion 113 are illustrated with dashed lines. However, the positions of the borders LAa and LBa and the borders LAb and LBb can be appropriately changed as long as the functions of the skin adhesive member 100 described hereafter are manifest within an allowable range. For example, the borders LAa, and LBa between the foundation portion 111 and the first extension portion 112 may be positioned further to the first extension portion 112 side than the example illustrated in FIG. 1, and may also be positioned more to the foundation portion 111 side.

The foundation portion 111 is positioned between the first extension portion 112 and the second extension portion 113 in the length direction Y. The foundation portion 111 functions as an anchor portion that supports each extension portion 112 and 113 that have been extended and adhered and also functions as a positioning portion when the tape body 110 of the skin adhesive member 110 is adhered to a body. The foundation portion 111 has one end portions 111Aa and 111Ba, and other end portions 111Ab and 111Bb in the length direction Y. In the example illustrated in FIG. 1, the foundation portion 111 has a polygonal shape (hexagonal shape), and the one end portions 111Aa and 111Ba are configured including two adjacent sides from among a plurality of sides of the polygonal shape. The other end potions 111Ab and 111Bb are configured including two sides opposing the two sides and the length direction Y included in the one end portions 111Aa and 111Ba. In the present embodiment, the foundation portion 111 is provided in line symmetry with regards to a central line C1 extending along the length direction Y on the tape body 110, and furthermore, is provided in line symmetry with regards to a central line C2 extending along the width direction X on the tape body 110. However, the foundation portion 111 may not be in line symmetry with the central lines C1 and C2.

The first extension portion 112 is formed extending in the length direction Y (Y-axis negative direction) from the one end portions 111Aa and 111Ba of the foundation portion 111, and is a portion adhered to the back side. The first extension portion 112 includes a pair of belt portions 112A and 112B provided arranges in the width direction X. Note that in the example illustrated in FIG. 1, only the pair of belt portions 112A and 112B are illustrated, but the first extension portion 112 may include three or more belt portions. The pair of belt portions 112A and 112B each make up a long, substantially rectangular shape extending in a straight line along the length direction Y. The pair of belt portions 112A and 112B are provided in line symmetry with, for example, the central line C1. However, the pair of belt portions 112A and 122B may not be in line symmetry with the central line C1.

The belt portion 112A includes a base end portion 112Aa, edge portions 112Ab and 112Ad, and a tip end portion 112Ac. The base end portion 112Aa is a connection portion with the one end portion 111Aa of the foundation portion 111. The edge portion 112Ab is a portion on the external side in the width direction in the belt portion 112A (in this case, the X-axis positive direction side), and the edge portion 112Ad is a portion on the internal side (in this case, the X-axis negative direction side). The edge portions 112Ab and 112Ad extend so as to be in parallel with the length direction Y. The tip end portion 112Ac is a portion on the tip end side (Y-axis negative direction side) in the belt portion 112A. The tip end portion 112Ac extends so as to be in parallel with the width direction X.

Similarly, the belt portion 112B includes a base end portion 112Ba, edge portions 112Bb and 112Bd, and a tip end portion 112Bc. The base end portion 112Ba is a connection portion with the one end portion of the 111Ba of the foundation portion 111. The edge portion 112Bd is a portion on the external side in the width direction X in the belt portion 112B (in this case, the X-axis negative direction side), and the edge portion 112Bb is a portion on the internal side (in this case, the X-axis positive direction side). The edge portions 112Bb and 112Bd extend so as to be in parallel with the length direction Y. The tip end portion 112Bc is a portion on the tip end side (Y-axis negative direction side) in the belt portion 112B. The tip end portion 112Bc extends so as to be in parallel with the width direction X.

As described above, the pair of belt portions 112A and 112B are provided in parallel arranged in the width direction X. In the example illustrated in FIG. 1, the pair of belt portions 112A and 112B are provided adjacent so that the edge portion 112Ad on the internal side of the belt portion 112A and the edge portion 112Bd on the internal side of the belt portion 112B contact. Furthermore, a slit is formed between the edge portion 112Ad on the internal side of the width direction X of the belt portion 112A and the edge portion 112Bd on the internal side of the width direction X of the belt portion 112B (position that overlaps the central line C1). Therefore, the belt portion 112A and the belt portion 112B are removable from each other at the slit portion when adhering. Note that the belt portion 112A and the belt portion 112B may be completely separated via the slit, but even if not completely separated, they may be removed easily via a perforation. In this manner, even if the pair of belt portions are not completely separated, it falls under “a belt portion provided aligned in the width direction X” when they are both divided in the width direction X in the first extension portion 112 via a separation portion such as a perforation. However, even before being adhered, the belt portion 112A and the belt portion 112B may be provided with a gap between them so that the edge portion 112Ad and the edge portion 112Bb do not contact.

The tape body 110 is processed (the fiber direction when manufacturing the tape body is adjusted) so that the elasticity in the length direction Y of the first extension portion 112 is higher than the elasticity in the width direction X. This is not limited to the first extension portion 112, but the entirety of the tape body 110 may be processed to facilitate extension in the length direction Y and inhibit extension in the width direction X.

The second extension portion 113 is formed extending in the length direction Y (Y-axis positive direction) from the other end portions 111Ab and 111Bb of the foundation portion 111, and is a portion adhered to the abdominal side. The shape of the second extension portion 113 is not necessarily limited, and can, for example, be the same shape as the first extension portion 112. In this case, as illustrated in FIG. 1, the second extension portion 113 includes pairs of belt portions 113A and 113B. The pair of belt portions 113A includes a base end portion 113Aa, edge portions 113Ab and 113Ad, and a tip end portion 113Ac, and the pair of belt portions 113B includes a base end portion 113Ba, edge portions 113Bb and 113Bd, and a tip end portion 113Bc. Because the components included in these pairs of belt portions 113A and 113B are the same as each component included in the pair of belt portions 112A and 112B, descriptions thereof are omitted.

As with the pair of belt portions 112A and 112B, the pair of belt portions 113A and 113B are provided in line symmetry with the central line C1 as the axis of symmetry. Here, if the foundation portion 111 is provided in line symmetry with the central lines C1 and C2 as the axis of symmetry as described above, the tape body 110 (in other words the skin adhesive member 100) can have a target shape in either the length direction Y or the width direction X. However, the pair of belt portions 113A and 113B may not be in line symmetry with the central line C1.

Next, the peeling sheet 120 will be described. As described above, the entire shape of the peeling sheet 120 is the same as the tape body 110. As illustrated in FIG. 2, a number of separation portions are formed on the peeling sheet 120 to make an easier peeling operation and adhering operation for the user. Note that the separation portions are parts that can be easily separated from other portions when the user peels a portion of the peeling sheet 120, may be formed by a slit that is completely cut, and may be formed by a perforation. In the example illustrated in FIG. 2, a plurality of separation portions 131A, 131B, 132A, 132B, 133A, 133B, 134A, and 134B are formed on the peeling sheet 120. Furthermore, by this, the peeling sheet 120 is divided into a plurality of regions 121, 122A, 122B, 123A, 123B, 124A, 124B, 125A and 125B.

The separation portions 131A and 131B are a pair of notches that are formed on the border region of the foundation portion 111 and the first extension portion 112, and separate the foundation portion 111 and the first extension portion 112. More specifically, from among the peeling sheet 120, the separation portions 131A and 131B are formed on the boundary region of the portion that protects the adhesive face of the foundation portion 111 (region 121) and the portion that protects the adhesive face of the first extension portion 112 (regions 122A, 122A, and the like), and cover the portion that protects the adhesive face of the foundation portion 111 and the portion that protects the adhesive face of the first extension portion 112.

Here, the boundary region of the foundation portion 111 and the first extension portion 112 refer to a region including the boundaries LAa and LBa of the foundation portion 111 and the first extension portion 112, and the vicinity thereof. The separation portions 131A and 131B may be formed on the boundaries LAa and LBa, and may be formed more to the foundation portion 111 side or to first extension portion 112 side than the boundaries LAa and LBa. For example, even if the separation portions 131A and 131B are formed only in the first extension portion 112, it is said that the separation portions 131A and 131B are formed on the boundary region of the foundation portion 111 and the first extension portion 112 if they are adjacent to the boundaries LAa and LBa. Note that as described above, because the position of the boundaries LAa and LBa may be changed as long as the functions of the skin adhesive member 100 are manifest within an allowable range, the position of the separation portions 131A and 131B can also be appropriately changed for the same purpose.

The separation portions 131A and 131B are inclined in the width direction X so that the portion on the external side in the width direction X is positioned more to the foundation portion 111 side than the portion on the internal side in the width direction X. In the example illustrated in FIG. 1, the separation portion 131A and the separation portion 131B are formed so that the inclining directions are different. Specifically, from among the pair of separation portions 131A and 131B, the separation portion 131A is inclined in the width direction X so that the portion on the external side in the width direction X (in this case, the X-axis positive direction side) is positioned more to the foundation portion 111 side (Y-axis positive direction side) than the portion on the internal side in the width direction X (in this case, the X-axis negative direction side) when viewed from the separation portion 131A on the X-axis positive direction side. From among the pair of separation portions 131A and 131B, the separation portion 131B is inclined in the width direction X so that the portion on the external side in the width direction X (in this case, the X-axis negative direction side) is positioned more to the foundation portion 111 side (Y-axis positive direction side) than the portion on the internal side in the width direction X (in this case, the X-axis positive direction side) when viewed from the separation portion 131A on the X-axis negative direction side.

The separation portions 132A and 132B are formed on the tip end portion of the pair of belt portions 112A and 112B, and are the pair of separation portions that separate the tip end portions 112Ac and 112Bc (regions 123A and 123B) of the pair of belt portions 112A and 112B from other portions (regions 122A and 122B).

The separation regions 133A and 133B are formed on the boundary region of the foundation portion 111 and the pair of belt portions 113A and 113B, and are the pair of separation portions that separate the foundation portion 111 (region 121) and the second extension portion 113 (regions 124A, 124B, and the like). The separation portions 133A and 133B are also inclined in the width direction X as with the separation portions 131A and 131B.

The separation portions 134A and 134B are formed on the tip end portion of the pair of belt portions 113A and 113B, and are the pair of separation portions that separate the tip end portions 113Ac and 113Bc (regions 125A and 125B) of the pair of belt portions 113A and 113B from other portions (regions 124A, 124B, and the like).

The region 121 is divided by the separation portions 131A and 131B and the separation portions 133A and 133B, and protects the adhesive face of the foundation portion 111.

The regions 122A and 122B are divided by the separation portions 131A and 131B and the separation portions 132A and 132B, and are the pair of layers that protect the adhesive face of portions other than the tip end portions 112Ac and 112Bc from among the pair of belt portions 112A and 112B.

The regions 123A and 123B are divided by the separation portions 132A and 132B, and are the pair of layers that protect the adhesive face of the tip end portions 112Ac and 112Bc from among the pair of belt portions 112A and 112B.

The regions 124A and 124B are divided by the separation portions 133A and 133B and the separation portions 134A and 134B, and are the pair of layers that protect the adhesive face of portions other than the tip end portions 113Ac and 113Bc from among the pair of belt portions 113A and 113B.

The regions 125A and 125B are divided by the separation portions 134A and 134B, and are the pair of layers that protect the adhesive face of the tip end portions 113Ac and 113Bc from among the pair of belt portions 113A and 113B.

The skin adhesive member 100 described can be created by, for example, creating one sheet by adhering a peeling sheet on the entirety of the adhesive face of a processed woven fabric, and then die cutting that sheet. Each notch of the separation portion 131A and the like can be formed by a kiss cut when cutting.

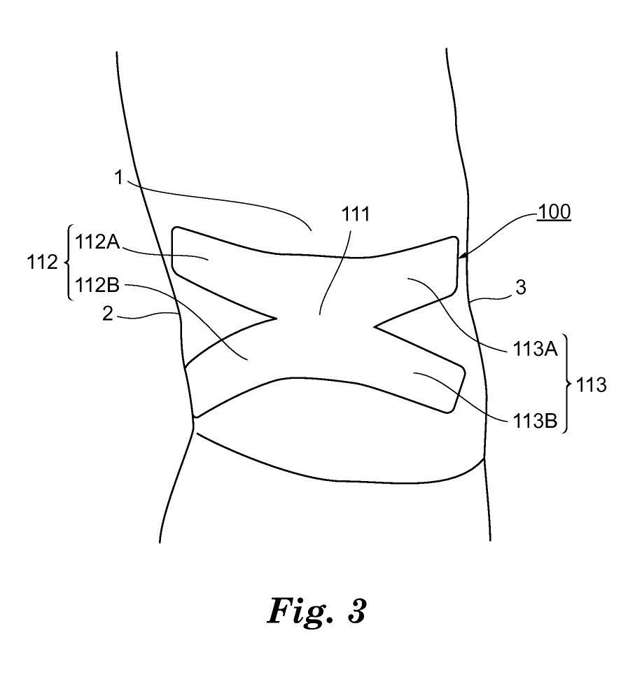

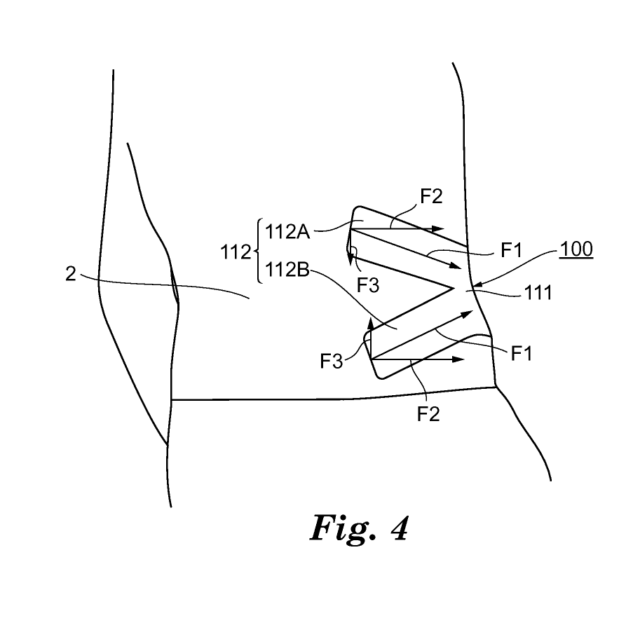

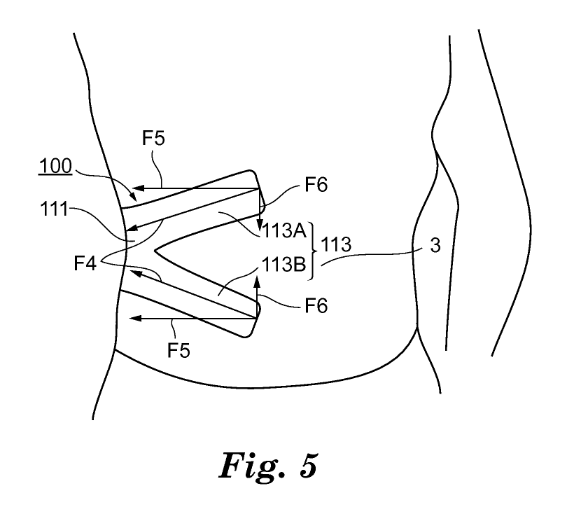

Next, an application example of the skin adhesive member 100 will be described with reference to FIGS. 3 to 5. FIGS. 3 to 5 are diagrams illustrating the skin adhesive member 100 adhered to the body of the user, where FIG. 3 is a diagram viewed from the side face of the adhering portion (side abdominal side of the user), FIG. 4 is a diagram viewed from the back face of the adhesive portion (back side of the user), and FIG. 5 is a diagram viewed from the front face of the adhesive portion (abdominal side of the user).

The user partially peels off the peeling sheet 120 exposing one portion of the adhesive face of the tape body 110 (adhesive face) and adheres the exposed portion in the appropriate location on the body of the user. Below, one example of an adhering procedure is shown. In the example shown below, a procedure is described where the foundation portion 111, the first extension portion 112, and the second extension portion 113 are adhered in that order, but the order of the adhering of the first extension portion 112 and the adhering of the second extension portion 113 may be reversed.

First, the user adheres the foundation portion 111 to the skin. Specifically, the user first peels the region 121 of the peeling sheet 120 off, and exposes the adhesive face of the foundation portion 111. Then, the user selects the direction of the skin adhesion material 100 so that the length direction of the skin adhesion material 100 is in a direction facing the back 2 side from the abdominal 3 side across the side abdomen, and adheres the exposed portion so that a central portion of the foundation portion 111 corresponds with the side abdominal portion on the upper portion of the pelvis.

Next, the user adheres the first extension portion 112. Specifically, the user peels off the regions 122A and 122B of the peeling sheet 120 exposing one portion of the adhesive face of the belt portions 112A and 112B, and adheres the exposed portion of the belt portions 112A and 112B to the back 2 side while holding the regions 123A and 123B. At this time, because the separation portions 131A and 131B that separate the regions 122A and 122B are inclined in the width direction, the user can easily adhere the exposed portion of the belt portions 112A and 112B diagonally. Furthermore, the user extends the belt portions 112A and 112B in the length direction (for example, 10 to 30 mm) by pulling the regions 123A and 123B toward, for example a substantially perpendicular direction from the inclination with the inclination of the separation portions 131A and 131B as a guide, and can adhere on the skin of the back 2 side so that the separation distance gets larger toward the tip end portion side in the width direction X. Furthermore, the user peels off the regions 123A and 123B of the peeling sheet 120 exposing the remaining portion of the adhesive face of the belt portions 112A and 112B, and adheres the tip end portions 112Ac and 112Bc of the belt portions 112A and 112B to the skin.

Lastly, the user adheres the second extension portion 113. Specifically, the user peels off the regions 124A and 124B of the peeling sheet 120 exposing one portion of the adhesive face of the belt portions 113A and 113B, and adheres the exposed portion of the belt portions 113A and 113B to the skin of the back 3 side while holding the regions 125A and 125B. At this time, as with the adhering of the belt portions 112A and 112B on the back 2 side, the belt portions 113A and 113B are extended in the length direction (for example, 10 to 30 mm), and the belt portions 113A and 113B can be adhered on the skin of the abdominal 3 side so that the separation distance gets larger toward the tip end portion side in the width direction X. Additionally, the user peels off the regions 125A and 125B of the peeling sheet 120 exposing the remaining portion of the adhesive face of the belt portions 113A and 113B, and adheres the tip end portions 113Ac and 113Bc of the belt portions 113A and 113B to the skin.

By the above procedure, as illustrated in FIGS. 3 to 5, the skin adhesive member 100 is adhered from the abdominal 3 side to the back 2 side across the side abdomen. The foundation portion 111 functions as the anchor portion adhered between the abdominal 3 and the back 2 (for 1 side abdomen). On the back 2 side, the pair of belt portions 112A and 112B are adhered diagonally to the standing direction (the direction the body extends in when standing) so that the separation distance gets larger toward the tip end portion side in the width direction. In other words, the first extension portion 112 has a V-shape that widens to the tip end portion with the first extension portion 112 adhered to the skin. Similarly, abdominal 3 side, the pair of belt portions 113A and 113B are adhered diagonally to the standing direction so that the separation distance gets larger toward the tip end portion 112Ac and 112Bc side in the width direction.

According to the above adhering procedure, because the skin adhesive member 100 is adhered extended in the length direction, stress such as that described next occurs on the skin adhesive member 100.

First, when describing the back 2 side with reference to FIG. 4, the pair of belt portions 112A and 112B are adhered extended in the length direction on the back 2 side. By being extended, stress occurs on the pair of belt portions 112A and 112B. In FIG. 4, the stress occurring on the pair of belt portions 112a is shown as stress F1 to F3 in the drawing.

The stress F1 is a force caused by the belt portions 112A and 112B trying to contract in the length direction, and operates in the length direction of the belt portions 112A and 112B. Because the belt portions 112A and 112B are adhered diagonally to the standing direction, the stress F1 occurs diagonally to the standing direction. Because of this, the stress F1 can be divided into a stress F2 that is a component operating in the horizontal direction (direction perpendicular to the standing direction) and a stress F3 that is a component that operates in the standing direction, and each of the stresses F2 and F3 perform a prescribed function.

The stress F2 supports the contracting of the muscles in the horizontal direction from the back 2 side to the abdominal 3 side. Meanwhile, the stress F3 operates so that the muscles in the standing direction (for example, standing muscles extending in the standing direction around the spine) are contracted. In other words, in the belt portion 112A, the stress F3 operates toward the lower side of the standing direction, and in the belt portion 112B, the stress F3 operates toward the upper side of the standing direction. By this, the stress F3 operates in a direction where the pair of belt portions 112A and 112B get closer, and support the contracting of muscles in the standing direction.

Next, when describing the abdomen 3 side with reference to FIG. 5, the pair of belt portions 113A and 113B are adhered extended in the length direction on the abdomen 3 side. By being extended, a stress F4 occurs on the pair of belt portions 113A and 113B.

By the stress F4, muscles on the abdomen 3 side are supported. For example, in the stress F4, a stress F5 that works in the horizontal direction supports the contracting of muscles of the horizontal direction from the abdomen 3 side toward the back 2 side, and a stress F6 that works in the standing direction supports the contracting of muscles of the standing direction.

Next, effects of the skin adhesive member 100 will be described. According to the skin adhesive member 100, because the second extension portion 113 is adhered on the abdomen 3 side, the muscles of the abdomen 3 side can be supported. In the example illustrated in FIG. 5, the stress F4 (including components of the stresses F5 and F6) occurs by the second extension portion 113. By this, muscles of the abdomen such as abdominal oblique muscles are supported and abdominal pressure can be increased. By increasing abdominal pressure, burden on the abdominal muscles is decreased and furthermore, for example, the weight of organs such as those of the digestive system can be supported.

Furthermore, according to the skin adhesive member 100, because the first extension portion 112 is adhered on the back 2 side, the muscles of the abdomen 3 side in addition to the back 2 side can be supported. Specifically, in the example illustrated in FIG. 4, the stress F1 (including components of the stresses F2 and F3) occurs on the back 2 side by the pair of belt portions 112A and 112B included in the first extension portion 112 being adhered to the back 2 side. Particularly, in the stress F1, the contracting of standing muscles can be supported by the stress F3 that operates in the standing direction. By the supporting of the contracting of the standing muscles, it is easier to support the user standing (extending posture opposite the bending posture).

Here, as a comparative example, the following problems occur with a skin adhesive member with the first extension portion 112 removed from the structure of the present embodiment. In other words, in the skin adhesive member as a comparative example, when the extending of the pair of belt portions 113A and 113B included in the second extension portion 113, or the opening degree (angle with regards to the horizontal direction) are not appropriate and the stresses F5 and F6 are too large, it is easier to have a bending posture, and it may be harder for the user to maintain an appropriate posture. In contrast to this, with the skin adhesive member 100, the stress F1 (including components of the stresses F2 and F3) occur by the first extension portion 112 being adhered to the back 2 side as described above. Because of this, for example, even when the force that tries to contract the abdominal oblique muscles on the abdominal 3 side by the stress F4 generated in the abdominal 3 side is too large, the force that tries to contract the abdominal oblique muscles on the back side 2 that is the opposite side of the abdominal 3 side sandwiching the foundation portion 111 operated by the stress F1 occurring on the back 2 side, thereby causing balance between the forces of the abdominal 3 side and the back 2 side. Therefore, it is easier for the user to maintain an appropriate posture.

Furthermore, the separation portion 131 inclined in the width direction is formed on the peeling sheet 120 that protects the adhesive face of the skin adhesive member 100. By this, it becomes simple to adhere so that the separation distance of the pair of belt portions 112A and 112B spreads. Furthermore, the user extends the pair of belt portions 112A and 112B at an appropriate angle following the inclination by pulling the region 123 toward, for example a substantially perpendicular direction from the inclination with the inclination of the separation portion 131 as a guide, and can adhere on the skin of the back 2 side.

Furthermore, in the peeling sheet 120, separation portions 132A and 132B that separate the tip end portions 112Ac and 112Bc of the pair of belt portions 112A and 112B and other portions are formed on the tip end portion of the pair of belt portions 112A and 112B. By this, the user can adhere the pair of belt portions 112A and 112B with a portion of the tip end side (regions 123A and 123B) remaining from the separation portions 132A and 132B in the peeling sheet. As a result, the user can perform an adhering operation without touching the adhesive face of the tip end portions 112Ac and 112B of the pair of belt portions 112A and 112B. By this, the adhesiveness of the adhesive faces being damaged can be avoided before the tip end portions 112Ac and 112B are adhered.

In addition, according to the skin adhesive member 100, the compression of, for example, abdominal horizontal muscles, can be supported as abdominal muscles other than abdominal oblique muscles. Specifically, back abdominal horizontal muscles (a portion of the back 2 side) are supported by the first extension portion 112 with the foundation portion 111 as an anchor portion, and front abdominal horizontal muscles (a portion of the abdominal 3 side) can be supported by the second extension portion 113. By supporting both the front and back abdominal horizontal muscles, a compression effect can be increased. Furthermore, when the skin adhesive member 100 has a target shape in the length direction and the width direction, the pair of belt portions 112A and 112B, and the pair of belt portions 113A and 113B can be adhered spread radially (for example, in an X-shape) sandwiching the foundation portion 111 as an anchor portion. Expansion in various directions can occur during exercise and the like, but when the pair of belt portions 112A and 112B and the pair of belt portions 113A and 113B are adhered spread radially, pressure is nearly evenly distributed without favoring a particular direction. By this, burden on the abdominal horizontal muscles can be reduced.

Furthermore, when the skin adhesive member 100 has a target shape in one of the length direction and the width direction, the adhering direction of the entire skin adhesive member 100 may be opposite the length direction Y and/or the width direction X, and thereby the user can use without worrying about the adhering direction of the entire skin adhering material 100.

Furthermore, when the entire skin adhesive member 100 has a substantially rectangular shape when die cutting and manufacturing the skin adhesive member 100 from one sheet as described above, (many) skin adhesive members 100 can be die cut and manufactured effectively from one sheet.

### Embodiment 2

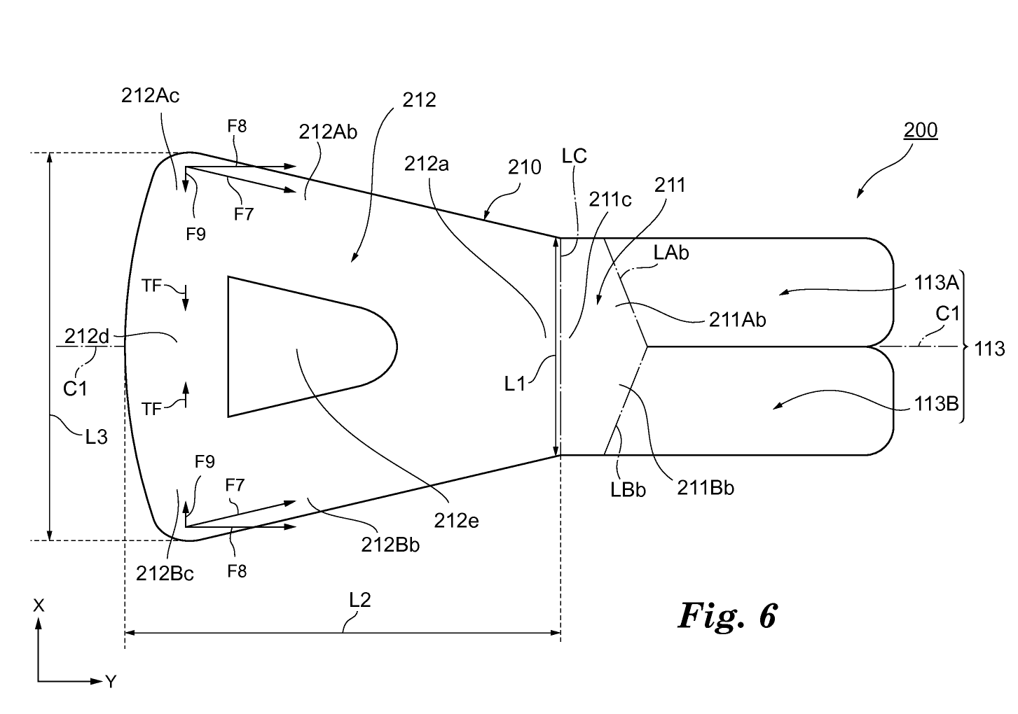

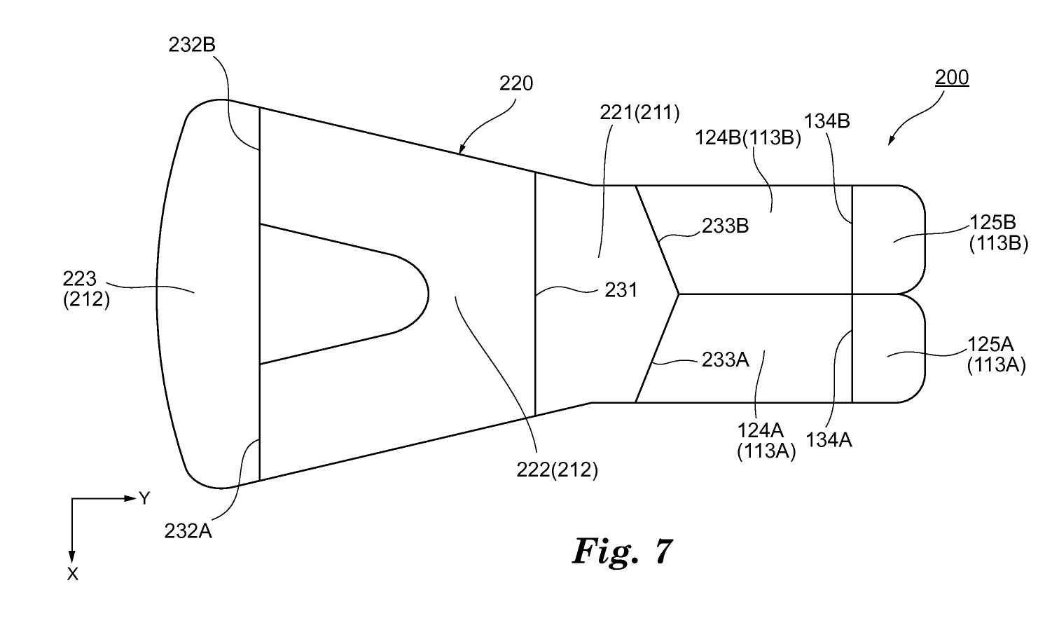

Next, the configuration of a skin adhesive member 200 according to embodiment 2 will be described with reference to FIGS. 6 and 7. FIG. 6 is a front view of the skin adhesive member 200, and FIG. 7 is a back view of the skin adhesive member 200. FIGS. 6 and 7 illustrate the skin adhesive member 200 before adhering to the body of the user. Embodiment 2 has a different shape of the first extension portion side compared to embodiment 1, and this is what will be particularly described. Because the shape of the second extension portion side is the same as embodiment 1, details thereof will be omitted.

The skin adhesive member 200 is made up of a double configuration by a tape body 210 and a peeling sheet 220. The tape body 210 and the peeling sheet 220 are the same shape entirely.

The tape body 210 extends along the length direction Y. The tape body 210 is provided with three portions of a foundation portion 211, a first extension portion 212, and the second extension portion 113 along the length direction Y. The second extension portion 113 was previously described with reference to FIG. 1, so the description thereof will be omitted. In FIG. 6, a border LC of the foundation portion 211 and the first extension portion 212 are illustrated with dashed lines. However, the position of the border LC can be appropriately changed as long as the functions of the skin adhesive member 200 are manifest within an allowable range. For example, the border LC of the foundation portion 211 and the first extension portion 212 may be positioned further to the first extension portion 212 side than the example illustrated in FIG. 6, and may also be positioned more to the foundation portion 211 side. The border of the foundation portion 211 and the second extension portion 113 are illustrated with dashed lines as borders LAb and LBb as with FIG. 1.

The foundation portion 211 is positioned between the first extension portion 212 and the second extension portion 113 in the length direction Y. As with the foundation portion 111 (FIG. 1), the foundation portion 211 also functions as an anchor portion that supports each extension portion 212 and 113 that have been extended and adhered and also functions as a positioning portion when the tape body 210 of the skin adhesive member 200 is adhered to a body. The foundation portion 211 has one end portion 211c, and other end portions 211Ab and 211Bb in the length direction Y. In the example illustrated in FIG. 6, the foundation portion 211 has a polygonal shape (pentagonal shape), and the one end portion 211c is configured including one side from among a plurality of sides of the polygonal shape. The other end potions 211Ab and 211Bb are configured including two sides of a side included in the one end portion 211c and a side opposing the length direction Y. In the present embodiment, the foundation portion 211 is provided in line symmetry with regards to a central line C1 extending along the length direction Y on the tape body 210. However, the foundation portion 211 may not be in line symmetry with the central line C1.

The first extension portion 212 is formed extending in the length direction Y (Y-axis negative direction) from the one end portion 211C of the foundation portion 211, and is a portion adhered to the back side. The first extension portion 212 includes a base end portion 212a, edge portions 212Ab and 212Bb, tip end portions 212Ac and 212Bc, a coupling portion 212d, and a through-hole 212e. The base end portion 212a is a connection portion with the one end portion 211c of the foundation portion 211. The edge portions 212Ab and 212Bb are a pair of edge portions that oppose each other in the width direction X. The tip end portions 212Ac and 212Bc are portions of the tip end sides (Y-axis negative direction side) of the first extension portion 212, and are also a pair of tip end portions on the tip end side of the pair of edge portions 212Ab and 212Bb. The coupling portion 212d is a portion that couples the pair of edge portions 212Ab and 212Bb on the tip end portion of the first extension portion 212, and more specifically, is a portion that couples the pair of tip end portions 212Ac and 212Bc together. The through-hole 212e is formed on the first extension portion 212 on a region surrounded by the one end portion 211c of the foundation portion 211 (or the base end portion 212a of the first extension portion 212), the pair of edge portions 212Ab and 212Bb, and the coupling portion 212b. The through-hole 212e can be formed by, for example, die cutting.

As with embodiment 1, in embodiment 2 as well, the tape body 210 is processed so that the elasticity in the length direction Y of the first extension portion 212 is higher than the elasticity in the width direction X.

In embodiment 2, the pair of edge portions 212Ab and 212Bb have a prescribed length L2 in the length direction Y. Furthermore, the pair of edge portions 212Ab and 212Bb, have separation distances in the width direction X that are larger towards the tip end portions 212Ac and 212Bc of the first extension portion 212. By this, the entirety of the pair of edge portions 212Ab and 212Bb have a V-shape widening to the tip end portion side. In addition, when including the base end portion 212a and the coupling portion 212d, the external shape of the first extension portion 212 has a trapezoidal shape with the base end side short and the tip end portion side long. The entirety of the tape body 210 including the foundation portion 211 and the second extension portion 133 (skin adhesive member 200) has a Y-shape widening to the tip end portion side of the first extension portion 212. The tape body 210 has a fixed length of the width direction X on a portion from the second extension portion 113 to the foundation portion 211, and with the boundary LC as the starting point, the length of the width direction X becomes longer from there toward the tip end side of the first extension portion 212.

Furthermore, the first extension portion 212 has a prescribed length L1 in the width direction X on the base end portion 212a, and has a prescribed length L3 in the width direction X on the tip end portions 212Ac and 212Bc. The shape of the first extension portion 212 is characterized by the dimensions of L1 to L3. In embodiment 2, L2 may be longer than L3. By making L2 longer, the area of the adhesive material adhered to the back side can be increased, and the supporting of the muscles on the back side can be improved. For example, it is preferable for the ratio of L2 to L1 (L2/L1) to be 1.5 or more, 3.0 or more, or 5.0 or more. It is preferable for the ratio of L2 to L3 (L2/L3) to be 1.0 to 2.5. It is preferable for the ratio of L3 to L1 (L3/L1) to be 1.4 or more, 2.0 or more, or 3.0 or more. As specific numerical values, for example, L1 can be 30 to 150 mm, L2 can be 75 to 250 mm, and L3 can be 75 to 250 mm.

Next, the peeling sheet 220 will be described. As described above, the entire shape of the peeling sheet 220 is the same as the tape body 210. As with the peeling sheet 120 (FIG. 2), a plurality of separation portions are formed on the peeling sheet 220. Specifically, a plurality of separation portions 231, 232A, 232B, 233A, 233B, 134A, and 134B are formed on the peeling sheet 220, and the peeling sheet 220 is divided into a plurality of regions 221 to 223, 124A, 124B, 125A and 125B. The separation portions 134A and 134B and the regions 124A, 124B, 125A, and 125B have been described previously with reference to FIG. 2, so a description thereof will be omitted.

The separation portion 231 is formed on the border region of the foundation portion 211 and the first extension portion 212, and separates the foundation portion 211 and the first extension portion 212. In the example illustrated in FIGS. 6 and 7, the separation portion 231 is in the vicinity of the boundary LC and is formed more on the first extension portion 212 side than the boundary LC. It can be said that in this state as well, the separation portion 231 is formed on the border region of the foundation portion 211 and the first extension portion 212. Note that in the example illustrated in FIG. 7, the separation portion 231 is formed along the width direction X, but the separation portion 231 may be inclined with regards to the width direction X. For example, the separation portion 231 may be configured diagonally such as the separation portions 431A and 431B described in Embodiment 4 described later.

The separation portions 232A and 232B are formed on the tip end portion of the first extension portion 212, and are the pair of separation portions that separate the tip end portions 212Ac and 212Bc of the first extension portion 212 and the coupling portion 212d (region 223) from other portions (region 222).

The separation regions 233A and 233B are formed on the boundary region of the foundation portion 211 and the pair of belt portions 113A and 133B, and are the pair of separation portions that separate the foundation portion 211 (region 221) and the second extension portion 113 (regions 124A, 124B, and the like). The separation portions 233A and 233B are also inclined in the width direction X as with the FIG. 2 separation portions 133A and 133B.

The region 221 is divided by the separation portion 231 and the separation portions 233A and 233B, and protects the adhesive face of the foundation portion 211.

The region 222 is divided by the separation portion 231 and the separation portions 232A and 232B, and protects the adhesive face of portions other than the tip end portions 212Ac and 212Bc, and the coupling portion 212d in the first extension portion 212.

The region 223 is divided by the separation portions 232A and 232B, and protects the adhesive face of the tip end portions 212Ac and 212Bc, and the coupling portion 212d of the first extension portion 212.

Next one example of an adhering procedure of the skin adhesive member 200 will be described. First, the user adheres the foundation portion 211 to the skin. Specifically, the user first peels the region 221 of the peeling sheet 220 off, and exposes the adhesive face (and one portion of the first extension portion 212) of the foundation portion 211. Then, the user selects the direction of the skin adhesion material 200 so that the length direction of the skin adhesion material 200 is in a direction facing the back side from the abdominal side across the side abdomen, and adheres the exposed portion so that a portion of the foundation portion 211 and the first extension portion 212 (boundary LC) correspond with the side abdominal portion on the upper portion of the pelvis.

Next, the user adheres the first extension portion 212. Specifically, the user peels off the region 222 of the peeling sheet 220 exposing one portion of the adhesive face of the first extension portion 212, and adheres the exposed portion of the first extension portion 212 to the skin of the back 2 side while holding the region 223. At this time, by pulling the region 223, the first extension portion 212 is extended in the length direction (for example, 10 to 30 mm), and adhered to the skin of the back side. Furthermore, the user peels off the regions 223 of the peeling sheet 220 exposing the remaining portion of the adhesive face of the first extension portion 212, and adheres the tip end portions 212Ac and 212Bc of the first extension portion 212, and the coupling portion 212d to the skin.

Lastly, the user adheres the second extension portion 113. The adhering procedure of the second extension portion 113 was previously described with reference to FIGS. 3 to 5, so the description thereof will be omitted.

By the above procedure, the skin adhesive member 200 is adhered from the abdominal side to the back side across the side abdomen. Here, because the pair of edge portions 212Ab and 212Bb, have separation distances in the width direction X that are larger toward the tip end portion side, the first extension portion 212 is adhered to the skin of the back side extended in the length direction on that shape. In this state, as with the pair of belt portions 112A and 112B (FIGS. 3 and 4) included in the first extension portion 112 of the skin adhesive member 100 according to embodiment 1, a V-shape is formed by the pair of edge portions 212Ab and 212Bb widening to the tip end portion side.

Because of this, even in the first extension portion 212 (at least near the pair of edge portions 212Ab and 212Bb) included in the skin adhesive member 200 adhered extended in the length direction, stress occurs that is the same as the stress that occurs in the first extension portion 112 included in the skin adhesive member 100 according to embodiment 1. In FIG. 6, the stress occurring on the first extension portion 212 when the skin adhesive member is extended is shown as stresses F7 to F9 in the drawing.

The stress F1 is a force caused by the first extension portion 212 trying to shrink in the length direction Y, and operates in a direction along the pair of edge portions 212Ab and 212Bb. The stress F7 occurs diagonally to the standing direction with the skin adhesive member 200 adhered. Because of this, the stress F7 can be divided into stress F8 that is a component that operates in the horizontal direction and the stress F9 that is a component that operates in the standing direction. These stresses F7 to F9 correspond to each stress F4 to F6 (FIG. 4) that occur in the skin adhesive member 100 according to embodiment 1, and operate the same.

Next, effects of the skin adhesive member 200 will be described. As described above, even in the first extension portion 212 included in the skin adhesive member 200, stress (F7 to F9) occurs that is the same as the stress (F4 to F6) that occurs in the first extension portion 112 included in the skin adhesive member 100 according to embodiment 1. Therefore, abdominal oblique muscles on the back side are supported and it can be easier for the user to maintain an appropriate posture, even with the skin adhesive member 200.

Moreover, in the skin adhesive member 200, the pair of edge portions 212Ab and 212Bb of the first extension portion 212 are coupled by the coupling portion 212b on the tip end portion. Here, by making the elasticity of the first extension portion 212 in the width direction X as small as possible, it can be done so that the coupling portion 212d mostly does not expand in the width direction X. Because the width direction X is equal to the standing direction with the skin adhesive member 200 adhered, the coupling portion 212d mostly does not extend in the standing direction. By this, for example, when the user takes an extreme stooped position, force that hinders the stooped position (reaction force TF illustrated in FIG. 6) can be made to occur by the coupling portion 212d included in the first extension portion 212 adhered to the back side mostly not extending in the standing direction. Therefore, by this as well, it can be easier for the user to maintain an appropriate posture.

Additionally, by having the coupling portion 212d, the area of the adhesive material adhered in a direction along the standing muscles can be increased, and the supporting of standing muscles can be improved more than without the coupling portion 212d. Furthermore, if there is no coupling portion 212d, because the skin between the pair of tip end portions 212Ac and 212Bc is not fixed to the skin adhesive member 200, the stress F9 in the standing direction is lessened by the expanding of the skin and not sufficiently communicated to the standing muscles, and support to the standing muscles may be insufficient. In contrast to this, by having the coupling portion 212d, the skin of this portion (in other words the portion between the pair of tip end portions 212Ac and 212Bc) is fixed to the skin adhesive member 200. By this, stress in the standing direction can be surely communicated to the standing muscles, and the supporting of the standing muscles can be improved.

Moreover, by the through-hole 212e being formed on the first extension portion 212, the adhering area of the skin adhesive member 200 can be reduced. By this, followability to the skin of the adhesive material can be improved.

### Embodiment 3

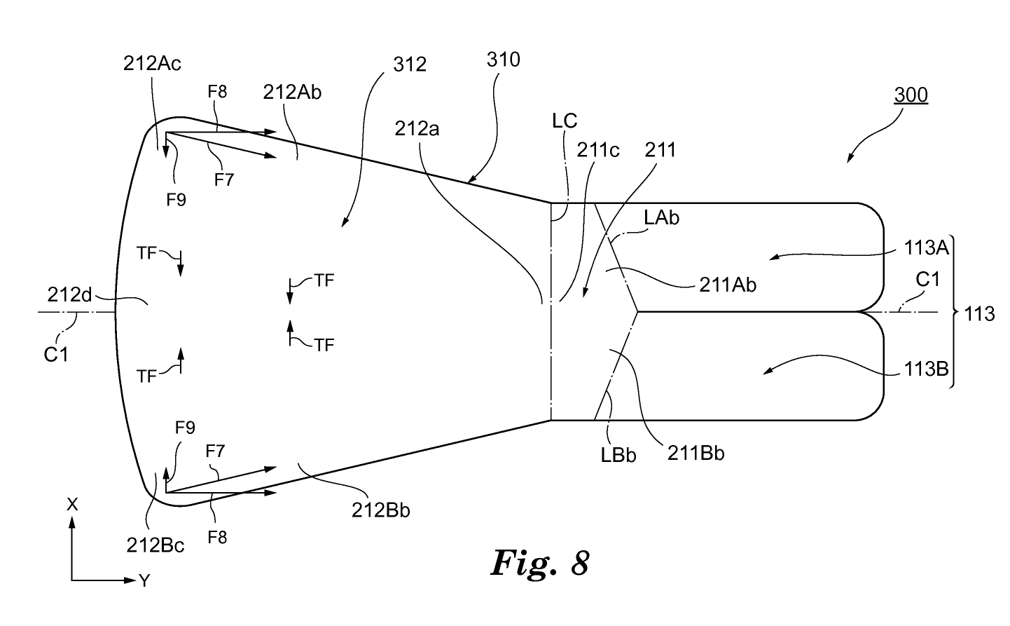

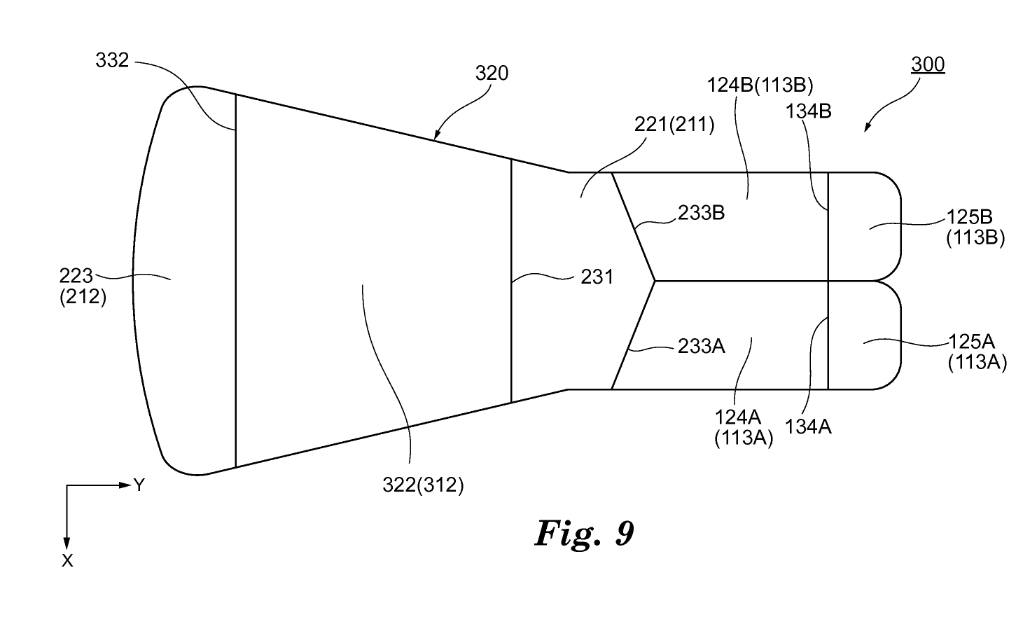

Next, the configuration of a skin adhesive member 300 according to embodiment 3 will be described with reference to FIGS. 8 and 9. FIG. 8 is a front view of the skin adhesive member 300, and FIG. 9 is a back view of the skin adhesive member 300. FIGS. 8 and 9 illustrate the skin adhesive member 300 before adhering to the body of the user. The skin adhesive member 300 differs when compared to the skin adhesive member 200 (FIG. 6) in the fact that it does not have the through-hole 212e.

The skin adhesive member 300 is made up of a double structure by a tape body 310 and a peeling sheet 320. The tape body 310 and the peeling sheet 320 are the same shape entirely.

The tape body 310 differs when compared to the tape body 210 (FIG. 6) in the fact that it includes a first extension portion 312 instead of the first extension portion 212. In the first extension portion 312, the adhesive material spreads across the entirety of a region surrounded by the one end portion 211c of the foundation portion 211 (or the base end portion 212a of the first extension portion 312), the pair of edge portions 212Ab and 212Bb, and the coupling portion 212b. The entirety of the first extension portion 312 has a trapezoidal shape with the base end side short and the tip end portion side long.

The peeling sheet 320 has a shape matching the tape body 310. The peeling sheet 320 differs when compared to the peeling sheet 220 (FIG. 7) in the fact that it includes a separation portion 332 instead of the separation portions 232A and 232B, and includes a region 322 instead of the region 222. The separation portion 332 is formed on the tip end portion of the first extension portion 312, and separates the tip end portions 212Ac and 212Bc of the first extension portion 321 and the coupling portion 212d (region 223) from other portions (region 322). The region 322 is divided by the separation portion 231 and the separation portion 332, and protects the adhesive face of portions other than the tip end portions 212Ac and 212Bc, and the coupling portion 212d in the first extension portion 312.

The adhering procedure of the skin adhesive member 300 can be the same as the adhering procedure of the skin adhesive member 200 according to embodiment 2 described previously, and thereby the description thereof will be omitted.

The first extension portion 312 of the skin adhesive member 300 includes the pair of edge portions 212Ab and 212Bb, as with the first extension portion 212 of the skin adhesive member 200 according to embodiment 2. Because of this, even in the first extension portion 312 adhered extended in the length direction, the same stress F7 to F9 occurs that is in the first extension portion 212 included in the skin adhesive member 200 according to embodiment 2. Therefore, abdominal oblique muscles on the back side are supported and it can be easier for the user to maintain an appropriate posture, even with the skin adhesive member 300. Additionally, because the first extension portion 312 includes the coupling portion 212d, the same reaction force TF can occur that is in the first extension portion 212 included in the skin adhesive member 200 according to embodiment 2. Here, with the first extension portion 312, because the through-hole 212e like that in the skin adhesive member 200 is not formed and adhesive material is spread on that portion, the reaction force TF can occur even in this portion. By this, because the reaction force TF that occurs when the user takes an extreme stooped posture increases, it becomes even simpler for the user to maintain an appropriate posture. Moreover, because there is no need to form the through-hole 212e like that in the skin adhesive member 200, it is simpler to manufacture the adhesive material.

### Embodiment 4

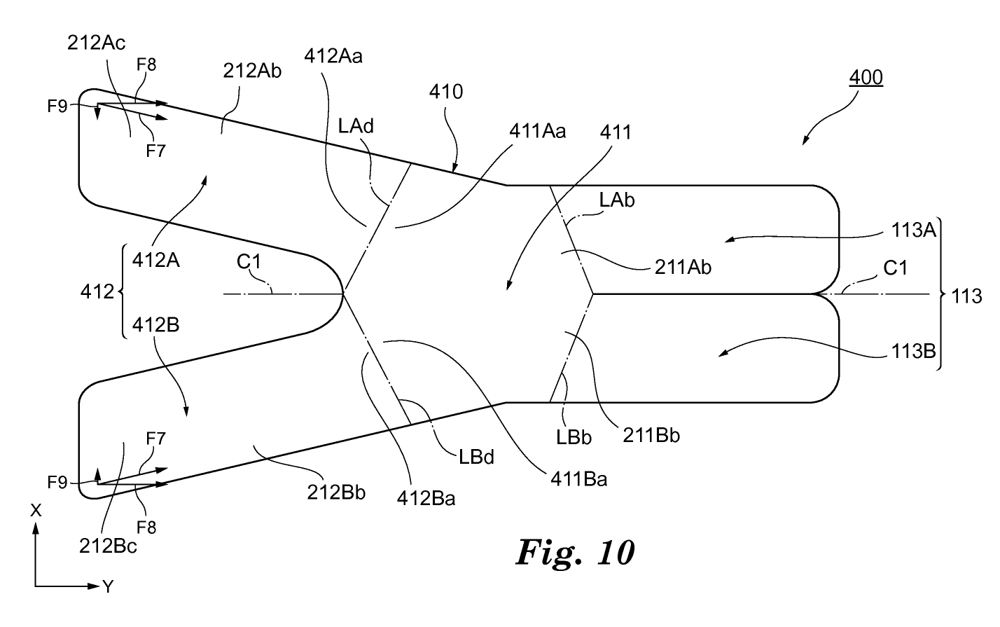

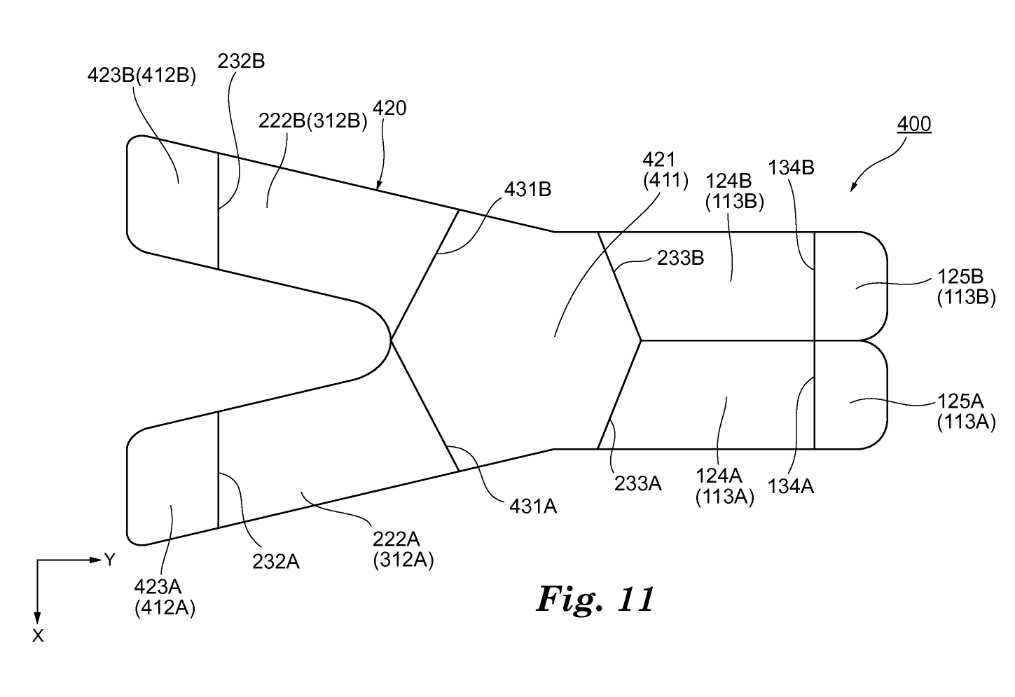

Next, the configuration of a skin adhesive member 400 according to embodiment 4 will be described with reference to FIGS. 10 and 11. FIG. 10 is a front view of the skin adhesive member 400, and FIG. 11 is a back view of the skin adhesive member 400. FIGS. 10 and 11 illustrate the skin adhesive member 400 before adhering to the body of the user. The skin adhesive member 400 differs when compared to the skin adhesive member 200 (FIG. 6) in the fact that it does not have the coupling portion 212d.

The skin adhesive member 400 is made up of a double structure by a tape body 410 and a peeling sheet 420. The tape body 410 and the peeling sheet 420 are the same shape entirely.

The tape body 410 differs when compared to the tape body 210 (FIG. 6) in the fact that it includes a foundation portion 411 and a first extension portion 412 instead of the foundation portion 211 and the first extension portion 212. In FIG. 10, borders LAd and LBd of the foundation portion 411 and the first extension portion 412 are illustrated with dashed lines. By the boundaries LAd and LBd, the first extension portion 412 is separated into a portion of belt portions of a belt portion 412A including the edge portion 212Ab and a belt portion 412B including the edge portion 212Bb, and by this, the foundation portion 411 is configured to have one end portions 411Aa and 411Ba in the length direction Y. The one end portions 411Aa and 411Ba of the foundation portion 411 are each configured to contact base end portions 412Aa and 412Ba of the belt portions 412A and 412B. The other end portions 211Ab and 211Bb of the foundation portion 411 were previously described with reference to FIG. 6, so the description thereof will be omitted. As with the foundation portion 111 (FIG. 1), the foundation portion 411 also functions as an anchor portion that supports each extension portion 412 and 113 that have been extended and adhered and also functions as a positioning portion when the tape body 410 of the skin adhesive member 400 is adhered to a body. Note that the position of the boundaries LAd and LBd are not limited to the example illustrated in FIG. 10, but can be appropriately changed as long as the functions of the skin adhesive member 400 are manifest within an allowable range. For example, the boundaries LAd and LBd may be configured as one boundary along the width direction X as with the boundary LC illustrated in FIG. 6. In this case, the shape of the foundation portion 411 is the same as the shape of the foundation portion 211 illustrated in FIG. 6, and the first extension portion 412 has a shape created by the edge portions 212Ab and 212Bb connected on the base end side.

The first extension portion 412 differs when compared to the first extension portion 212 (FIG. 6) in the fact that it does not have the coupling portion 212d. Additionally, because the foundation portion 411 and the first extension portion 412 are divided by the boundaries LAd and LBd and the belt portions 412A and 412B are formed, each of the belt portions 412A and 412B include the base end portions 412Aa and 412Ba. The first extension portion 412 has a V-shape widening to the tip end portion side.

The peeling sheet 420 has a shape matching the tape body 410. The peeling sheet 420 differs when compared to the peeling sheet 220 (FIG. 7) in the fact that it includes separation portions 431A and 431B instead of the separation portion 231, and includes regions 421 and 423A and 423B instead of the regions 221 and 223. The separation portions 431A and 431B are a pair of separation portions that separate the foundation portion 411 and the pair of belt portions 412A and 412B included in the first extension portion 412. The region 421 is divided by the separation portions 431A and 431B and the separation portions 233A and 233B, and protects the adhesive face of the foundation portion 411. The regions 423A and 423B are divided by the separation portions 232A and 232B, and protect the adhesive face of the tip end portions 212Ac and 212Bc of the first extension portion 412.

The adhering procedure of the skin adhesive member 400 can be the same as the adhering procedure of the skin adhesive member 200 described previously, and thereby the description thereof will be omitted.

The first extension portion 412 of the skin adhesive member 400 also includes the pair of edge portions 212Ab and 212Bb, as with the first extension portion 212 of the skin adhesive member 200 according to embodiment 2. Because of this, even in the first extension portion 412 adhered extended in the length direction, the same stress F7 to F9 occurs that is in the first extension portion 212 included in the skin adhesive member 200 according to embodiment 2. Therefore, abdominal oblique muscles on the back side are supported and it can be easier for the user to maintain an appropriate posture, even with the skin adhesive member 300.

The present invention is not limited to the embodiments described above. Configurations appropriately assembled with characteristic portions of each embodiment also apply to the embodiments of the current invention.

Furthermore, above, a description of a configuration of the second extension portion 113 adhered to the abdominal side including the pair of belt portions 113A and 113B, but the configuration of the second extension portion is not necessarily limited to this. For example, as a shape of the second extension portion, one belt shape may be used, or various shapes such as the first extension portions 212, 312, and 314 illustrated in FIGS. 6, 8, and 10 may be used.

### DESCRIPTION OF NUMERALS

1 . . . Side abdomen, 2 . . . Back, 3 . . . Abdomen, 100, 200, 300, 400 . . . Skin adhesive member, 110, 210, 310, 410 . . . Tape body, 111, 211, 411 . . . Foundation portion, 112, 212, 312, 412 . . . First extension portion, 113 . . . Second extension portion, 112A, 112B, 113A, 113B, 412A, 412B . . . Belt portion, 120, 220, 320, 420 . . . Peeling sheet.

1. A skin adhesive member extending in a length direction comprising:
a foundation portion having one end portion and an other end portion in the length direction,
a first extension portion extending in the length direction from the one end portion of the foundation portion,
a second extension portion extending in the length direction from the other end portion of the foundation portion, wherein
the first extension portion includes at least a pair of belt portions provided aligned in a width direction perpendicular to the length direction.

2. A skin adhesive member extending in a length direction comprising:
a foundation portion having one end portion and an other end portion in the length direction,
a first extension portion extending in the length direction from the one end portion of the foundation portion,
a second extension portion extending in the length direction from the other end portion of the foundation portion, wherein
the first extension portion has a prescribed length in the length direction and includes a pair of edge portions opposing each other in a width direction perpendicular to the length direction, and
the pair of edge portions have separation distances in the width direction that are larger towards a tip end portion side of the first extension portion.

3. The skin adhesive member according to claim 2, wherein the first extension portion includes a coupling portion that couples the pair of edge portions on the tip end portion.

4. The skin adhesive member according to claim 3, wherein a through-hole is formed on a region surrounded by the one end portion of the foundation portion, the pair of edge portions, and the coupling portion in the first extension portion.

5. The skin adhesive member according to claim 3, wherein an adhesive material is spread on the entirety of a region surrounded by the one end portion of the foundation portion, the pair of edge portions, and the coupling portion in the first extension portion.

6. The skin adhesive member according to claim 1, further comprising a peeling member that protects an adhesive face of the skin adhesive member, wherein
the peeling member includes a separation portion formed on the border region of the foundation portion and the first extension portion, and
the separation portion has a portion on the external side in the width direction positioned more to the foundation portion side than the portion on the internal side in the width direction.

7. The skin adhesive member according to claim 1, wherein the elasticity in the length direction of the first extension portion is higher than the elasticity in the width direction.

8. The skin adhesive member according to claim 2, further comprising a peeling member that protects an adhesive face of the skin adhesive member, wherein
the peeling member includes a separation portion formed on the border region of the foundation portion and the first extension portion, and
the separation portion has a portion on the external side in the width direction positioned more to the foundation portion side than the portion on the internal side in the width direction.

9. The skin adhesive member according to claim 2, wherein the elasticity in the length direction of the first extension portion is higher than the elasticity in the width direction.
